(19) United States
(12) Patent Application Publication
(10) Pub. No.: US 2007/0010584 A1
(43) Pub. Date: Jan. 11, 2007

(54) Compositions and methods for orthostatic intolerance

(76) Inventor: Stephen J. Peroutka, Burlingame, CA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/570,611

(22) PCT Filed: Sep. 1, 2004
(86) PCT No.: PCT/US04/28271

§ 371 Date: Mar. 3, 2006

Related U.S. Application Data
(60) Provisional application No. 60/499,726, filed on Sep. 4, 2003.

(51) Int. Cl. A61K 31/198; A61K 31/137

(52) U.S. Cl. 514/567; 514/649

(57) ABSTRACT

The present invention relates to compositions and methods for treating orthostatic intolerance in a subject in need thereof. Methods involve, e.g., administering an amount of a norepinephrine precursor, whereby said amount is effective for treating orthostatic intolerance. A useful norepinephrine precursor is threo-3-(3,4-dihydroxyphenyl) serine (L-threo-DOPS), a derivative thereof, or a pharmaceutically-acceptable salt thereof.

This application claims the benefit of U.S. Provisional Application No. 60/499,726, filed 4 Sep. 2003.

### BACKGROUND OF THE INVENTION

Orthostatic intolerance (OI) is a clinical syndrome consisting of group of symptoms that can occur after assuming an upright posture. Although a variety of OI definitions have been used in the medical literature, OI can best be defined as the development of lightheadedness or “dizziness”, as well as visual changes and other symptoms, upon arising from the supine position to an upright position. A key feature of OI is that all of these symptoms occur in the absence of a significant drop in systemic blood pressure (e.g., a reduction of systolic blood pressure of at least 20 mm Hg or as a reduction of diastolic blood pressure of at least 10 mm Hg within 3 minutes of standing from a supine position) that results in orthostatic hypotension (The Consensus Committee of the American Autonomic Society and the American Academy of Neurology. Consensus statement on the definition of orthostatic hypotension, pure autonomic failure, and multiple system atrophy. Neurology, 46:1470, 1996).

Many of the OI symptoms induced by postural change are listed in Table 1. Most of these symptoms appear to result from a mild to moderate reduction in cerebral blood flow. In addition, other OI symptoms may be based on cerebral hypoperfusion but might also result from peripheral nervous system dysfunction (e.g. tremulousness). An additional key feature of OI is that these posturally-induced symptoms can be relieved by resumption of the supine position (a clinical observation suggesting that enhancement of cerebral blood flow can relieve all of the symptoms of OI).

Clinical signs that frequently accompany the symptoms of OI are a significant postural tachycardia (i.e., >30 beats per minute) and significant postural increases in plasma norepinephrine concentrations (Jacob et al, Circulation 96:575, 1997; Shannon et al, N Engl J Med 342:541, 2000). OI has been reported to occur frequently in young women (Low et al, Neurology 45(Suppl 5):S19, 1995; Jacob et al, 1997; Goldstein et al, Circulation 106:2358, 2002) but it is also common in the general population (Junker et al, Neurology 60:331, 2003). OI can be quite disabling to patients when it is frequent and severe. OI has been reported to be worse in the morning, in hot weather, after exercise and after a large meal (Low et al, 1995).

TABLE 1
Clinical Symptoms of Orthostatic Intolerance

Syncope
Dizziness/lightheadness
Visual disturbances
Headache
Weakness, lethargy and fatigue
Exercise intolerance
Chest discomfort
Anxiety
Clamminess
Nausea
Tremulousness
Flushing
Palpitations
Cognitive Impairment (e.g., diminished concentration)
Shortness of breath
Heat Intolerance
Associated findings
Mitral valve prolapse
Irritable bowel syndrome
Chronic fatigue syndrome
Inflammatory bowel disease
Fibromyalgia

A large number of medical terms have been used to describe clinical conditions that are characterized, at least in part, by OI (Table 2). Syndrome names such as postural orthostatic tachycardia syndrome (POTS), soldier's heart and vasoregulatory asthenia all refer to symptom complexes that are the same or similar to OI. The specific cause of these putative disorders remains unknown or speculative.

TABLE 2
Syndromes Characterized by Orthostatic Intolerance of Unknown Etiology

Anxiety neurosis
Chronic Fatigue Syndrome
DaCosta syndrome
Effort Syndrome
Hyperadrenergic postural hypotension
Hyperdynamic beta-adrenergic state
Idiopathic hypovolemia
Idiopathic orthostatic intolerance
Idiopathic orthostatic tachycardia
Irritable heart
Migraine
Mitral valve prolapse syndrome
Myalgic encephalomyelitis
Neurocirculatory asthenia
Neuropathic Postural Tachycardia Syndrome
Orthostatic anemia
Orthostatic tachycardia
Panic Disorder
Partial dysautonomia
Postural Orthostatic Tachycardia Syndrome (POTS)
Postural Tachycardia Syndrome
Soldier's heart
Systolic click-late systolic click murmur syndrome
Sympathicotonic orthostatic hypotension
Sympathotonic orthostatic hypotension
Vasoregulatory asthenia

Moreover, a large number of seemingly unrelated clinical entities are characterized by OI (Table 3). These disorders are caused by specific pathophysiological disturbances (e.g., anemia) that affect the physiological function of the cardiovascular system. These disorders may all result in decreased cerebral perfusion in the upright position. When these symptoms occur in the absence of a significant change in systemic blood pressure, then they would be correctly classified as OI.

TABLE 3
Specific Clinical Conditions that May Cause Orthostatic Intolerance

Anemia
Bedrest
Bradury-Eggleston Syndrome
DBH deficiency
Hypovolemia
Multiple System Atrophy (MSA)
Parkinson's Disease
Pure Autonomic Failure (PAF)
Shy-Drager Syndrome
Spaceflight

Orthostatic Intolerance is Distinct from Orthostatic Hypotension

A clear distinction must be made between OI and orthostatic hypotension (OH) since these terms are sometimes used interchangeably (and therefore incorrectly) in the medical literature. Specifically, OH is a manifestation of abnormal blood pressure regulation due to various etiologies. Based on a consensus agreement, OH is a physical examination finding defined as a reduction of systolic blood pressure of at least 20 mm Hg or as a reduction of diastolic blood pressure of at least 10 mm Hg within 3 minutes of standing from a supine position (The Consensus Committee of the American Autonomic Society and the American Academy of Neurology, 1996). Occasional OH patients may not experience a drop in blood pressure until they stand for at least 10 minutes.

Thus, OH is a physical examination finding and not a symptom complex or disease state. Confusion in the medical literature arises because OH may be associated with various symptoms that are similar, or even identical, to the symptoms of OI. However, OH often exists in the absence of any OI symptoms. Indeed, no statistical association has been observed between the presence of OI and OH in the adult population (Junker et al, 2003). Therefore, the symptom complex of OI is clinically distinct from the physical examination finding of OH (Low et al, 1995).

In fact, by the definition used in the present report, OI patients cannot have OH. Once again in contrast to assumptions found sometimes in the medical literature, postural blood pressure changes do not reliably predict the symptoms of cerebral hypoperfusion (i.e., OI) (Low et al, Am J Med Sci 317:124, 1999). Moreover, whereas OH is associated with reduced plasma norepinephrine levels, OI is associated with normal or elevated norepinephrine levels (Low et al, 1995). These important clinical and laboratory differences between OI and OH suggest that both their etiologies and their treatments may differ significantly (Low et al, 1995).

Prevalence of Orthostatic Intolerance

The prevalence of OI has not been studied extensively. It has been estimated that as many as 500,000 Americans may suffer from some form of OI (Robertson, Am J Med Sci 31:775, 1999). The fact that this unequivocal disorder is not better appreciated in clinical medicine has been attributed to a lack of “disease recognition” (Robertson, 1999). Another factor limiting the clinical recognition of this syndrome is that it has historically been viewed as a psychosomatic illness in many patients. In addition, the symptoms of OI are often dismissed entirely by physicians because of their nonspecific nature.

Only a single prevalence study of OI can be identified in the medical literature. A random sample of adult residents (age≧45) in Olmsted County, Minnesota was surveyed for the presence of OI (Junker et al, 2003). OI was defined in this study as the presence of lightheadedness with upright posture. Using a self-administered questionnaire subjects were asked the following questions:
(1) Have you experienced lightheaded spells?
(2) If yes, did the symptoms occur in the upright posture?
(3) Have you ever experienced a black-out (fainting, complete loss of consciousness)?

Of the 4203 subjects invited to participate in the study, 2042 (47%) participated (Junker et al, 2003). Of these 2042 participants, 1597 responded to question (1), 1557 to (2), and 1649 to (3). Of the 1597 subjects responding to (1), 42% (672/1597) reported experiencing spells of lightheadedness. Of the 1557 subjects responding to (2), 30% (468/1557) reported symptoms of OI. Of these 468 OI subjects, 13.7% reported symptoms frequently (i.e., daily 3.3%, weekly 5.7% or monthly 4.8%). OI was associated with a slightly lower mean systolic blood pressure (i.e., 129.8 vs. 132.1 m Hg; (p=0.04) and slightly lower mean age (i.e., 60.6 vs. 62.4 years; p<0.001). A history of syncope was reported by 30% of subjects with OI vs. 15% of subjects with no OI (p<0.001). There was no association between the prevalence of OI and gender (p=0.86), body mass index (p=0.64), diabetes (p=0.33), history of hypertension (p=0.78), coronary artery disease (p=0.75) or orthostatic hypotension (p=0.12).

These authors concluded that OI is a common symptom complex with a prevalence of 30% in this community of persons aged ≧45 years (Junker et al, 2003). 14% of OI subjects report symptoms frequently (daily, weekly, or monthly), representing 4% of the population studied. Since there are ˜100 M Americans older than 45 years of age, frequent OI may affect ˜4 M Americans in this age group.

Clearly, OI represents a common symptom complex within the senior adult population. The fact that OI was not associated with OH suggests that different pathophysiological processes may underlie the two clinical conditions.

The Current Treatment of Orthostatic Intolerance

To date, there have been no known controlled studies to evaluate therapeutic approaches to OI. Indeed, only a few investigators have made recommendations for the therapeutic management of OI (Low et al, 1995; Jacob et al, 1997; Jacob and Biaggioni, Am J Med Sci 317:88, 1999). Due to a variety of issues such as an inconsistent and confusing nomenclature, heterogeneity of patient subgroups and contradictory clinical observations, effective treatment regimens have not been developed for many patients with OI. A summary of currently recommended therapies is listed below.

Physical Countermeasures

A variety of non-pharmacological measures can ameliorate OI. For example, patients should be encouraged to change posture slowly. Sleeping with the head of the bed raised may relieve daytime symptoms by promoting sodium retention and reducing nocturnal diuresis (Low et al, 1995). Patients should avoid prolonged standing and support hose may be of slight benefit to some individuals. Regular modest-intensity exercise can increase overall vascular tone and reduce venous pooling and should, therefore, be recommended to patients with OI.

Volume Expansion

Expansion of plasma volume can occur easily by increasing oral sodium intake. In the absence of heart failure, sodium intake can be increased 5 to 10 g above the usual dietary level by liberally salting food or taking sodium chloride tablets.

Saline infusions can also rapidly increase plasma volume. In a study of 13 patients with OI (who also had elevated norepinephrine levels and orthostatic tachycardia), normal saline (I L given IV over 1 hour) decreased both supine and standing heart rate (Jacob et al, 1997). It as been proposed that volume expansion causes the baroreceptors to reflexively lower sympathetic tone (Jacob et al, 1997), a theory that assumes OI is caused by excessive sympathetic tone.

Sodium-retaining hormones such as fludrocortisone can also be used (Goldstein et al, 2002). 9-α-Fludrocortisone (0.1 to 0.5 mg/day orally) can improve the peripheral vasoconstrictor response to sympathetic stimulation but is effective only when sodium ingestion is adequate. Recombinant erythropoeitin has also been recommended for certain patients with OI (Jacob and Biaggioni, 1999).

Alpha1-Adrenergic Receptor Agonists (i.e., Sympathomimetics)

Midodrine is an alpha1-adrenergic receptor agonist that has a 4-hour duration of action (half-life=30 minutes). It does not cross the blood brain barrier. In a study of 13 patients with OI (who also had elevated norepinephrine levels and orthostatic tachycardia), midodrine (5-10 mg PO) decreased both supine and standing heart rate (Jacob et al, 1997). It has been proposed that midodrine acts to attenuate baroreceptor activation, thus lowering sympathetic tone (Jacob et al, 1997). Other sympathomimetics such as ergotamine and phenylpropamolamine have been recommended but without any known clinical trial data (Jacob and Biaggioni, 1999).

Alpha2-Adrenergic Receptor Agonists (i.e., Central Sympatholytics)

Central sympatholytic drugs such as clonidine (an alpha2-adrenergic receptor agonist) have been recommended for patients with OI and significant increases in upright plasma norepinephrine levels (Jacob and Biaggioni, 1999). However, in a study of 13 patients with OI (who also had elevated norepinephrine levels and orthostatic tachycardia), clonidine (0.1 mg PO) decreased both supine and standing heart rate but also significantly decreased blood pressure (Jacob et al, 1997). In this study, the OI patients were reported to tolerate clonidine poorly (Jacob et al, 1997).

Beta-Adrenergic Receptor Antagonists

Propranolol was the first drug used to treat a syndrome consisting of OI and orthostatic tachycardia and it has been reported to be effective in a number of patients (Frohlich et al, 1966; Goldstein et al, 2002). Beta-adrenergic receptor blockade with propranolol leads to unopposed alpha-adrenergic peripheral vascular vasoconstriction and can therefore prevent the vasodilatation that occurs in some patients upon standing (Jacob and Biaggioni, 1999). Propranolol therapy has also been reported to enhance the beneficial effects of sodium and mineralocorticoid therapy in some patients with OI.

### DESCRIPTION OF THE INVENTION

The present invention relates to compositions and methods for treating orthostatic intolerance in a subject in need thereof. Methods involve, e.g., administering an amount of a norepinephrine precursor to a subject in need thereof, whereby said amount is effective for treating orthostatic intolerance.

The precursor is administered in amounts that are effective to treat orthostatic intolerance. The term “treating” is used conventionally, e.g., the management or care of a subject for the purpose of combating, alleviating, reducing, relieving, improving, etc., one or more of the symptoms associated with orthostatic intolerance, including those described in Table 1. Treating with a norepinephrine precursor also can reduce the frequency, severity, intensity, and/or duration of any of the aforementioned symptoms.

Any norepinephrine precursor that is effective in treating orthostatic intolerance can be used. A norepinephrine precursor comprises any compound that is converted into norepinephrine. These include, e.g., a substrate of the enzyme dopa decarboxylase that can be converted to norepinephrine, such as threo-3-(3,4-dihydroxyphenyl)serine (threo-DOPS), or a substrate of the enzyme dopamine beta-hydroxylase that can be converted to norepinephrine, such as dopamine. The norepinephrine precursor can be used for the synthesis of norepinephrine, and therefore can increase its levels in the synaptic terminals.

The present invention also relates to methods of treating orthostatic intolerance in a subject in need thereof, comprising, administering to the subject an amount of threo-3-(3,4-dihydroxyphenyl) serine, a derivative thereof, or a pharmaceutically-acceptable salt thereof, which is effective to treat orthostatic intolerance.

As explained below, for a number of reasons, it was unexpected that a norepinephrine precursor would be useful in treating orthostatic intolerance. Independent of its pathophysiological cause, it is clear that the major debilitating symptoms of orthostatic intolerance (i.e., dizziness, visual changes, fatigue and syncope) can be attributed directly to intraparenchymal (i.e., the intrinsic brain vasculature) cerebral hypoperfusion. The regulation of cerebral blood flow is critical for brain function since it ensures an adequate and stable oxygen supply to the central nervous system.

Cranial blood flow is divided into two major subdivisions, with each modulated by a different control system. Important anatomical and physiological distinctions exist between the regulation of intraparenchymal and extraparenchymal cranial blood flow. Specifically, one major vascular subdivision of cranial blood flow supplies the brain tissue directly and arises from the internal carotid and vertebral arteries. The intrinsic brain vasculature is under the direct control of local metabolic and myogenic factors. For example, increases in arterial carbon dioxide levels result in arterial vasodilatation within the brain, whereas hyperventilation leading to low levels of carbon dioxide result in vasoconstriction within the brain.

The second major vascular subdivision of the head arises predominantly from the external carotid artery and supplies the external portion of the head and the surface of the brain (i.e., the meninges). This subdivision has no direct impact on intraparenchymal blood flow. The sympathetic nervous system (SNS) plays a major role in the regulation of cranial blood flow to this vascular subdivision (Harper et al, Arch Neurol. 27(1):1-6, 1972; Hernandez-Perez et al, Stroke 6(3):284-292, 1975). SNS innervation of the external carotid artery and its branches is extremely dense, while SNS innervation is essentially absent in the vascular distribution of the internal carotid and vertebral arteries (i.e., the intraparenchymal cranial blood flow).

While sympathetic stimulation causes significant vasoconstriction in the vascular distribution of the external carotid artery, it has little effect on the diameter of the internal carotid vasculature (Harper et al, Arch Neurol. 27(1): 1-6, 1972; Spira et al, Neurology 28(2):162-173, 1978; Vasquez and Pinardi, Gen Pharmacol. 23(4):775-780, 1992; Michalicek et al, J Cereb Blood Flow Metab. 16(3):507-516, 1996). As a result, it was not anticipated that a norepinephrine precursor could be utilized to treat symptoms associated with intraparenchymal cerebral hypoperfusion. Moreover, the use of a norepinephrine precursor, such as L-threo-DOPS, for orthostatic intolerance appears to be against the existing conventional theories. For example, the frequently observed increase in plasma norepinephrine levels in OI appears to argue against the use of DOPS in this disorder (Frohlich et al, Arch Intern Med. 117(5):614-619, 1966; Narkiewicz and Somers, Circulation 98(20):2105-2107, 1998; Shannon et al, N Engl J Med. 342(8):541-549, 2000; Goldstein et al, Circulation 106(18):2358-2365, 2002).

The amount of the norepinephrine precursor can be effective in increasing, augmenting, enhancing, etc., the intraparenchymal cranial blood flow, thereby treating symptoms associated with orthostatic intolerance. By reducing the occurrence of intraparenchymal cerebral hypoperfusion, the frequency and severity of the condition is treated. Although it is believed that the mechanism relates to the sympathetic nervous system, the present invention relates to the use of a norepinephrine precursor to treat orthostatic intolerance, regardless how the therapeutic effect is achieved.

The specific dose level and frequency of dosage may vary, depending upon a variety of factors, including the activity of the specific active agent, its metabolic stability and length of action, rate of excretion, mode and time of administration, the age, body weight, health condition, gender, diet, etc., of the subject, and the severity, intensity, and frequency of the symptoms associated with orthostatic intolerance. A precursor in accordance with the present invention can be immediately effective in achieving therapeutic efficacy, or it can reach its maximal effect after multiple or regularly administered doses, e.g., one or more doses a day for a week, two weeks, a month, etc.

Any patient can be treated for OI in accordance with the present invention, including, e.g., patients displaying one or more symptoms shown in Table 1, or any of the syndromes described in Tables 2 and 3. Patient groups can also be excluded from the present invention, including any of those mentioned in Tables 2 and 3, as well as patients who have orthostatic hypotension (e.g., experience a drop in blood pressure as defined earlier).

Threo-3-(3,4-dihydroxyphenyl)serine (also known as threo-DOPS or DOPS or droxidopa) is a synthetic amino acid precursor of NE (Freeman R., Clin. Neuropharm., 14, 296-304, 1991). DOPS is directly converted to NE via L-aromatic amino acid decarboxylase (AADC), also known as dopa decarboxylase (DDC). It has four stereoisomers, L-threo-DOPS, D-threo-DOPS, L-erythro-DOPS, and D-erythro-DOPS. Of the four, L-threo-DOPS is preferred, but a racemate can also be used. Peak plasma levels of DOPS occur about 3 hours after oral ingestion, whereas peak NE levels occur about 5 hours after ingestion. Increased plasma levels of both molecules remain at least 12 hours after oral administration of DOPS (Suzuki et al., Eur. J. Clin. Pharmacol., 23(5):463-8, 1982). Specific uptake of DOPS has also been demonstrated in microvessel preparations (Hardebo et al., Acta Physiol Scand., 107(2):161-7, 1979). Although threo-3-(3,4-dihydroxyphenyl)serine is known as a norepinephrine precursor, the present invention includes any therapeutic effect for orthostatic intolerance, regardless of its mechanism of action or how it is achieved.

DOPS has been used to treat motor or speech disturbances (e.g., U.S. Pat. No. 5,656,669), Parkinson's disease, cerebral ischemia (e.g., EP 887 078), urinary incontinence (e.g., U.S. Pat. No. 5,266,596), orthostatic hypotension (Freeman, 1991), and pain (e.g., U.S. Pat. No. 5,616,618; EP 681 838).

Any effective amount of threo-3-(3,4-dihydroxyphenyl)serine can used, e.g., from about 10 mg to about 600 mg per day, about 50 mg to about 150 mg per day, etc. Effective amounts can be determined routinely, and may vary depending upon the age, health, gender, and weight of a patient, as well as the severity, frequency, and duration of OI. Amounts can be administered in a multiple doses over the course of the day, e.g., in order to achieve a prophylactic effect.

Threo-3-(3,4-dihydroxyphenyl) serine can be prepared according to any suitable method. These processes include those described in, e.g., U.S. Pat. Nos. 4,480,109, 4,562,263 and 5,864,041. It can be used as a racemic or optically active isomer, e.g., L-threo-DOPS.

Pharmaceutically-acceptable salts of threo-3-(3,4-dihydroxyphenyl)serine can also be used, including addition salts, e.g., inorganic acids, such as hydrochloric acid, hydrobromic acid, and sulfuric acid, and organic acids, such as fumaric acid, citric acid, tartaric acid, and succinic acid.

Any pharmacologically active derivative of threo-3-(3,4-dihydroxyphenyl)serine can be used. These include, e.g., N-methyl-3-(3,4-dihydroxyphenyl)serine alkyl esters, such as N-methyl-D,L-threo-3-(3,4-dihydroxyphenyl)serine and N-methyl-L-threo-3-(3,4 dihydroxyphenyl)serine, lower alkyl esters, methyl esters, ethyl esters, n-propyl esters, isopropyl esters, etc., as described in U.S. Pat. No. 5,288,898.

Precursors can be further combined with any other suitable additive or pharmaceutically acceptable carrier. Such additives include any of the substances already mentioned, as well as any of those used conventionally, such as those described in Remington: The Science and Practice of Pharmacy (Gennaro and Gennaro, eds, 20th edition, Lippincott Williams & Wilkins, 2000); Theory and Practice of Industrial Pharmacy (Lachman et al., eds., 3rd edition, Lippincott Williams & Wilkins, 1986); Encyclopedia of Pharmaceutical Technology (Swarbrick and Boylan, eds., 2nd edition, Marcel Dekker, 2002).

These are generally referred to herein as “pharmaceutically acceptable carriers” to indicate they are combined with the active drug and can be administered safely to a subject for therapeutic purposes. These include, but are not limited to, antioxidants, preservatives, dyes, tablet-coating compositions, plasticizers, inert carriers, excipients, polymers, coating materials, osmotic barriers, devices and agents which slow or retard solubility, etc.

The precursors can be in any suitable form, without limitation. Forms suitable for oral use, include, but are not limited to, tablets, troches, lozenges, aqueous or oily suspensions, dispersible powders or granules, emulsions, hard or soft capsules, solutions, syrups and elixirs. Compositions intended for oral use may be prepared according to any method known to the art for the manufacture of pharmaceutical compositions.

Compositions comprising precursors can also be formulated for controlled release, where release of the active ingredient is regulated or modulated to achieve a desired rate of delivery into the systemic circulation A controlled release formulation can be pulsed, delayed, extended, slow, steady, immediate, rapid, fast, etc. It can comprise one or more release formulations, e.g. extended- and immediate-release components.

Any suitable dosing interval can be used in accordance with the present invention. Extended delivery systems can be utilized to achieve a dosing internal of once every 24 hours, once every 12 hours, once every 8 hours, once every 6 hours, etc. The dosage form/delivery system can be a tablet or a capsule suited for extended release, but a sustained release liquid or suspension can also be used. A controlled release pharmaceutical formulation can be produced which maintains the release of, and or peak blood plasma levels of, threo-3-(3,4-dihydroxyphenyl) serine, derivative thereof, or salt thereof, over a period of at least 6 hours, 12 hours, 18 hours, 24 hours, etc. With this type of formulation, the DOPS can be continuously released in such a way that it is available and effective for maintaining the nerve terminal pools of norepinephrine.

Norepinephrine precursors in accordance with the present invention can be administered in any form by any effective route, including, e.g., oral, parenteral, enteral, intraperitoneal, topical, transdermal (e.g., using any standard patch), ophthalmic, nasally, local, non-oral, such as aerosal, spray, inhalation, subcutaneous, intravenous, intramuscular, buccal, sublingual, rectal, vaginal, intra-arterial, and intrathecal, etc. It can be administered alone, or in combination with any ingredient(s), active or inactive.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever. The entire disclosure of all patents and publications cited herein are hereby incorporated by reference in their entirety, including U.S. Provisional Application No. 60/499,726, filed 4 Sep. 2003.

What we claim:

1. A method of treating orthostatic intolerance in a subject in need thereof, comprising:
administering an amount of a norepinephrine precursor, whereby said amount is effective for treating orthostatic intolerance.

2. A method of claim 1, wherein said norepinephrine precursor is L-threo-DOPS.

3. A method of claim 1, wherein said amount is effective to increase sympathetic terminal levels of norepinephrine.

4. A method of claim 1, wherein said amount is effective for increasing intraparenchymal blood flow to the brain.

5. A method of claim 1, wherein said amount is effective for reducing intraparenchymal cerebral hypoperfusion.

6. A method of claim 1, wherein said orthostatic intolerance is associated with anemia, bedrest, Bradury-Eggleston Syndrome, DBH deficiency, hypovolemia, Multiple System Atrophy, Parkinson's disease, Pure Autonomic Failure, Shy-Drager Syndrome, or spaceflight.

7. A method of claim 1, wherein said orthostatic intolerance is associated with postural tachycardia.

8. A method of claim 1, wherein said subject does not experience orthostatic hypotension.

9. A method of treating orthostatic intolerance in a subject in need thereof, comprising:
administering an amount of a norepinephrine precursor, whereby said amount is effective for treating orthostatic intolerance, with the proviso that said subject does not have orthostatic hypotension.

10. A method of claim 9, wherein said norepinephrine precursor is L-threo-DOPS.

11. A method of claim 9, wherein said amount is effective to increase sympathetic terminal levels of norepinephrine.

12. A method of claim 9, wherein said orthostatic intolerance is associated with anemia, bedrest, Bradury-Eggleston Syndrome, DBH deficiency, hypovolemia, Multiple System Atrophy, Parkinson's disease, Pure Autonomic Failure, Shy-Drager Syndrome, spaceflight.
